(19) United States
(12) Patent Application Publication
(10) Pub. No.: US 2026/0315407 A1
(43) Pub. Date: Oct. 15, 2026

(54) CONTINUOUS PROCESS LNP FORMULATION

(71) Applicant: Arranta Bio Holdings, LLC, Watertown, MA (US)

(72) Inventors: Edita Botonjic-Sehic, Watertown, MA (US); Syed Kaschif Ahmed, Watertown, MA (US); Mark Jandreski, Watertown, MA (US); Hossein Hamedi, Watertown, MA (US); Marianna Sofman, Watertown, MA (US); Busuyi Adebayo, Watertown, MA (US); Aaron Cowley, Watertown, MA (US); Nisha A. Patel, Watertown, MA (US); Rikki Kaufman, Watertown, MA (US); Aula Al Muslim, Watertown, MA (US)

(21) Appl. No.: 19/636,625

(22) Filed: Apr. 1, 2026

Related U.S. Application Data
(60) Provisional application No. 63/781,539, filed on Apr. 1, 2025.

(51) Int. Cl. A61K 9/51; A61K 31/7105; A61K 47/24; G01N 21/65; G16C 20/10

(52) U.S. Cl. CPC A61K 9/5192; A61K 9/5123; A61K 31/7105; A61K 47/24; G01N 21/65; G16C 20/10

(57) ABSTRACT

The present invention provides apparatus, systems, and methods for continuous manufacturing of RNA.

### CROSS-REFERENCE TO RELATED APPLICATION

This application claims the benefit of U.S. Provisional Patent Application No. 63/781,539 filed on Apr. 1, 2025. The subject matter of the aforementioned patent application is incorporated by reference herein in their entireties.

### TECHNICAL FIELD

The present invention relates to improved process analytic technology (PAT) and methods for use in a continuous pharmaceutical and biopharmaceutical manufacturing systems which utilize lipid nanoparticle formulation of a therapeutic drug product.

### BACKGROUND

Conventional biopharmaceuticals are manufactured using batch production methods in which human intervention is required to process a set quantity of material to be produced at the same time. Batch operations may require as long as 1-2 months or more from bioreactor to final formulated product. An alternative approach is continuous manufacturing, which is attractive due to its potential to reduce costs while increasing productivity and improving product consistency. Continuous manufacturing processes have been developed in the chemical, petrochemical, food, and mechanical industries. In these contexts, continuous processes have demonstrated less reliance on human labor and fewer gaps in transitioning between unit operations in the process resulting in increased productivity. Further, a smaller facility footprint required by a continuous process reduces facility costs.

There is a need for continuous manufacturing systems in the biopharmaceutical sector, as an alternative to the more time consuming, resource intensive, and expensive batch processes that represent the current standard of practice, as acknowledged by regulatory agencies that have urged the adoption of continuous biomanufacturing in this sector. See National Academies of Sciences, Engineering and Medicine. Continuous manufacturing for the modernization of pharmaceutical production. 2019.

While continuous bioprocessing has yet to be fully realized, several innovations in unit operations have made its implementation more feasible in biopharmaceutical manufacturing. Despite these and other advances in adapting various unit operations to continuous processing, significant challenges remain in process integration, real-time monitoring, and control systems. Currently, RNA manufacturing is performed in a series of batch processes including lipid preparation, LNP processing, a series of purification and concentration steps, excipient addition, sterile filtration, and final fill and finish of the drug product. There is a need to develop systems and methods for monitoring, modeling, and refinement of continuous manufacturing of RNA. These systems will embrace developing a fully integrated and continuous RNA process including adoption of novel process analytical technologies and data science techniques. The present invention addresses this need by providing process analytic technology and methods for continuous processing of unit operations involved in lipid nanoparticle formulation.

### BRIEF SUMMARY

Provided are an apparatus, system and methods for in-line continuous monitoring and control of a lipid nanoparticle formulation process, including methods for determining a lipid concentration in a process fluid. The apparatus, system and methods are also useful for determining certain product critical quality attributes, or “CQAs” including mRNA concentration, total lipid concentration and component lipid concentration.

In one aspect, provided is a compact modular apparatus that includes a Raman spectrometer and optionally an optical detector selected from one or both of a dynamic light scattering (DLS) detector and a multiangle light scatter (MALS) detector, a tubing assembly, an optional container external to the Raman spectrometer, a pump, and a controller that includes at least one soft sensor configured to receive multivariate Raman spectra data from the spectrometer and transform the data into a CQA selected from one or more of mRNA concentration, total lipid concentration, and component lipid concentration. In aspects, the component lipid concentration includes a concentration value for one or more of a phospholipid, such as distearoylphosphatidylcholine (DSPC), cholesterol, including Phytochol™, an ionizable cationic lipid, such as a C12-200, DLin-MC3-DMA, SM-102, or ALC-0315, and/or a polyethylene glycol (PEG) lipid derivative, such as a C14-PEG2000. The soft sensor may include a predictive model for estimating the product CQA from the received Raman spectra data. The apparatus may also include a DLS detector and/or a MALS detector and associated soft sensors adapted to receive and transform DLS and MALS data into a CQA. The apparatus may also include a near-IR spectrometer and/or a mid-IR spectrometer and associated soft sensor adapted to receive and transform IR spectral data into a CQA. In aspects, the CQA may be selected from one or more of lipid nanoparticle size, size distribution, and polydispersity index (PDI, d) of encapsulated mRNA or a sucrose concentration value. In aspects, the CQA measured by IR may include sucrose and/or ethanol. In aspects, the model is calibrated using off-line analytical measurements. Other technical features may be readily apparent to one skilled in the art from the following figures, descriptions, and claims.

Also provided is a system including an apparatus as described herein and four sequential unit operations, CP1, CP2, CP3, and CP4, where CP1 is a lipid mixing operation, CP2 is a lipid nanoparticle formulation operation in which a drug product is mixed with the lipids of CP1 to form drug product encapsulated in lipid nanoparticles, CP3 is a tangential flow filtration operation and CP4 is an excipient formulation operation in which excipients are added to produce the final drug product formulation. The system may also include one or more digital twins configured to trend and predict product attributes and process parameters at one or more of the unit operations. Other technical features may be readily apparent to one skilled in the art from the following figures, descriptions, and claims.

Also provided are methods. In one aspect, provided is a method for in-line continuous monitoring and control of a lipid nanoparticle formulation process, where the method includes acquiring a set of Raman spectral data from a process fluid at a lipid mixing unit operation, CP1, transforming the Raman spectral data, via operation of a pre-established computational model of a soft sensor, into a lipid concentration value, comparing, via operation of a controller, the lipid concentration value to a predetermined range of lipid concentration values preloaded into the controller, and performing an operation, via the controller, selected from hold and release of the process fluid from CP1 to the next unit operation in the lipid nanoparticle formulation process, CP2. The process fluid may include a mixture of lipids and the lipid concentration value may be selected from a total lipid concentration value and a concentration value of one or more component lipids in the mixture. The one or more component lipids may include one or more of a phospholipid, such as distearoylphosphatidylcholine (DSPC), an ionizable cationic lipid, such as a C12-200, DLin-MC3-DMA, SM-102, and ALC-0315, and/or a polyethylene glycol (PEG) lipid derivative, such as a C14-PEG2000. The set of Raman spectral data may include data of raw Raman spectra in a region of 1200-1800 cm⁻¹ and the lipid concentration value may be a total lipid concentration value. The Raman spectral data may also include data of raw Raman spectra in a region of 1200-1800 cm⁻¹ and in a region of 2700-3000 cm⁻¹ and the lipid concentration value may be a concentration value of one or more component lipids in the mixture. The method may also include where the computational model utilizes characteristic bands at 1419 cm⁻¹, 1323 cm⁻¹, and 1671 cm⁻¹ to determine a concentration value of the ionizable lipid, ALC-0315, and/or cholesterol. The method may also include where the computational model utilizes a partial least squares (PLS) regression algorithm. The method may further include creating, by a processor of a digital twin, a model of the lipid mixing unit operation, CP1.

Also provided is a method for determining a lipid concentration in a process fluid, where the method includes acquiring a set of Raman spectral data from a process fluid that includes a lipid or a mixture of lipids, and transforming the Raman spectral data, via operation of a pre-established computational model, into one or more lipid concentration values, thereby determining the concentration of the lipid (or lipids) in the process fluid. In aspects where the process fluid includes a mixture of lipids, the lipid concentration value is selected from a total lipid concentration value and a concentration value of one or more component lipids in the mixture. The method may also include where the one or more component lipids is one or more of a phospholipid, such as distearoylphosphatidylcholine (DSPC), an ionizable cationic lipid, such as a C12-200, DLin-MC3-DMA, SM-102, and ALC-0315, and/or a polyethylene glycol (PEG) lipid derivative, such as a C14-PEG2000. In aspects, the set of Raman spectral data includes data of raw Raman spectra in a region of 1200-1800 cm⁻¹ and the lipid concentration value is a total lipid concentration value. In aspects, the set of Raman spectral data includes data of raw Raman spectra in a region of 1200-1800 cm⁻¹ and in a region of 2700-3000 cm⁻¹ and the lipid concentration value is a concentration value of one or more component lipids in the mixture. The method may also include where the computational model utilizes characteristic bands at 1419 cm⁻¹, 1323 cm⁻¹, and 1671 cm⁻¹ to determine a concentration value of the ionizable lipid, ALC-0315, and/or cholesterol. The method may also include where the computational model utilizes a partial least squares (PLS) regression algorithm. In aspects, the method further includes creating, by a processor of a digital twin, a model of the lipid mixing unit operation, CP1. The method may also include where the computational model utilizes an entirety of the regions identified (i.e. 1200-1800 cm⁻¹ and 2700-3000 cm⁻¹) to develop a model for total lipid concentration, where individual lipid component concentrations utilize characteristic bands incorporating the regions identified from a loadings analysis in which: characteristic bands of DSPC are at 2901 cm⁻¹, characteristic bands of cholesterol are at 2933 cm⁻¹, 1419 cm⁻¹, and 1671 cm⁻¹, characteristic bands of ionizable lipid are at 1323 cm⁻¹, 2831 cm⁻¹, and 2901 cm⁻¹, and characteristic bands of PEG are at 1700 cm⁻¹. The method may also include trending and predicting, by a processor of the digital twin, the lipid mixing unit operation, CP1. Other technical features may be readily apparent to one skilled in the art from the following figures, descriptions, and claims.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers, with redundant description omitted. For purposes of clarity and simplicity, not every element is labeled in every figure, nor is every element of each embodiment shown where illustration is not necessary to allow those of ordinary skill in the art to understand the disclosure.

To easily identify the discussion of any particular element or act, the most significant digit or digits in a reference number refer to the figure number in which that element is first introduced.

FIG. 1 is a block diagram illustrating a continuous process to provide a bulk drug product 110 in accordance with one embodiment.

FIG. 2 is a block flow diagram illustrating a method to monitor a continuous RNA process in accordance with one embodiment.

FIG. 3 illustrates a PAT box connected to a process vessel in accordance with one embodiment.

FIG. 4 illustrates modeling of total lipid concentration from Raman spectra acquired from samples containing known concentrations of lipid.

FIG. 5A shows reference lipid concentration (mg/ml) as measured by HPLC versus the model prediction for a calibration set.

FIG. 5B shows reference lipid concentration as measured by HPLC versus the model prediction for a cross-validation set.

FIG. 5C shows lipid concentration as measured by HPLC versus the model prediction for a drug product in CP1 during two independent runs.

FIG. 6A illustrates the raw Raman spectra of LNPs produced with a T-mixer, following a freeze-thaw cycle, in a focused area from 1200-1800 cm⁻¹ for a series of dilutions ranging from 1 to 0.2× of a stock solution.

FIG. 6B shows the same spectra following pre-processing.

FIG. 6C shows the reference concentration versus model prediction for the calibration set.

FIG. 6D shows the reference concentration versus model prediction for the cross-validation set.

FIG. 7A shows the raw spectra of dry powder lipid components including the ionizable lipid, cholesterol, distearoylphosphatidylcholine (DSPC), and a polyethylene glycol (PEG) lipid derivative.

FIG. 7B shows the pre-processed spectra where pre-processing included smoothing, 2nd derivation filter and standard normal variate normalization, but no baseline correction or subtraction.

FIG. 8A shows a selected Raman spectral region of from 1200-1800 cm⁻¹. Component lipid peaks appear at 1700 (DSPC, PEG), 1140 (Cholesterol) and 1673 (Cholesterol).

FIG. 8B shows a selected Raman spectral region of 2700-3000 cm⁻¹ containing the lipid vibrational signatures.

FIG. 9A shows a selected Raman spectral region from 1400-1500 cm⁻¹ with a peak at 1419 cm⁻¹ for the ionizable lipid (ALC) and cholesterol.

FIG. 9B shows a selected Raman spectral region from 1400-1500 cm⁻¹ with a peak at 1323 cm⁻¹ for the ionizable lipid.

FIG. 9C shows a selected Raman spectral region from 1400-1500 cm⁻¹ with a cholesterol peak at 1671 cm⁻¹.

FIG. 9D shows the information in FIG. 9A-9C integrated into a model spectra.

FIG. 10A shows a selected Raman spectral region from 2700-3000 cm⁻¹ with a peak at 2831 for ALC

FIG. 10B shows a selected Raman spectral region from 2700-3000 cm⁻¹ with a peak at 2933 for cholesterol.

FIG. 10C shows a selected Raman spectral region from 2700-3000 cm⁻¹ with a peak at 2901 for ALC and DSPC.

FIG. 10D shows the information in FIG. 10A-10C integrated into a model spectra.

FIG. 11 shows a heatmap summarizes Q² values (the cross-validated coefficient of determination), a stringent quantification of model performance, across 24 model variants for the total lipid concentration prediction in the LSP pool unit operations.

FIG. 12 shows a summary of partial least square (PLS) models.

FIG. 13 shows a Raman shift of process runs.

FIG. 14 illustrates alignment of preprocessed model spectra.

FIG. 15 illustrates a table of model performance using the prediction spectra from a process run.

FIG. 16 shows another summary of PLS models.

FIG. 17 shows a reference concentration versus model prediction for the cross-validation set for the best performing model.

### DETAILED DESCRIPTION

The present invention provides modular apparatuses used in the production of LNP formulated drug products and methods for monitoring and control of the LNP process. The LNP process includes unit operations such as lipid prep, LNP formulation, filtration, excipient addition, sterile filtration, and fill and finish. The processes and apparatuses may be combined to provide a system for continuous manufacturing of LNP encapsulated drug product. Continuous manufacturing can be achieved by closed loop control of unit operations via operation of soft sensors and controllers.

As used herein, the term “soft sensor” has its usual and customary meaning as a software-based sensor including a combination of input process data and a computational model that utilizes the process data to predict a target quantity. In accordance with aspects of the present invention, a soft sensor utilizes a partial least squares (PLS) regression algorithm and off-line analytics to generate predictive models for critical product quality attributes (CQAs) using Raman, dynamic light scatter (DLS), near infrared (near-IR), ultraviolet/visible (UV/VIS), or Fourier-transform infrared (FTIR) data. FTIR may also be referred to as “mid-IR”. In aspects, the input data used for the PLS model are multivariate signals generated from Raman spectra and the CQA is a concentration value of a particular lipid, or of total lipids, or of mRNA in a process fluid comprising lipids and/or lipid nanoparticles. In other aspects, the input data are multivariate signals generated from dynamic light scattering (DLS) and/or multi-angle light scattering (MALS) detection instruments. Calibration of the PLS regression models is performed using off-line analytical measures to ensure specificity of the models to the biomolecules of interest. In general, the PLS models utilize principal component analysis (PCA) to identify parameters contributing to the most variations in the Raman spectral data (X space), which are then correlated to the off-line reference values, e.g., HPLC measurements (Y space).

The term “controller” refers to a controlling device coupled with process analytic technology, also referred to herein as a “PAT instrument”, which can include spectroscopic instruments such as a Raman, ultraviolet/visible (UV/VIS), near-infrared (near-IR), or Fourier-transform IR (FTIR, mid-IR) spectrophotometer, or an instrument for measuring light scatter, such as a MALS or DLS instrument, where the controller is in communication with a unit operation of the LNP process, or with a hold vessel located in fluid communication between two unit operations. The controller receives converted biochemical parameter values transferred from the PAT instrument and compares the received values with corresponding preset parameter values input to the controller in advance. Based on results from the comparing action, the controller automatically controls an aspect of the unit operation and/or releases a process pool held at the hold vessel. In aspects, PAT instruments may be modelled via models executed by an advanced process controller of the PAT box and/or by a remote computer.

Current pharmaceutical and biomanufacturing processes that include lipid nanoparticle (LNP) formulation are not optimized for cost, labor, process development, yield, quality, safety, or equipment performance. For example, filtration membrane failures and downtime are difficult to monitor, predict, and avoid as are excipient addition pump failures and downtime. Conventional systems are unable to provide real-time feedback control and require considerable time for drug products to be released for fill and finish. The industry further has been unable to develop suitable processes and methods for in-process monitoring of LNP profiles. The present invention addresses these deficiencies in the manufacture of LNP encapsulated drug products by supporting a continuous manufacturing process in which CQAs are monitored in-line and integrated into process control and real-time release protocols. CQAs are determined by in-line spectroscopic measurements, for example by Raman spectroscopy and light scattering, including dynamic light scattering (DLS) and multi-angle light scattering (MALS).

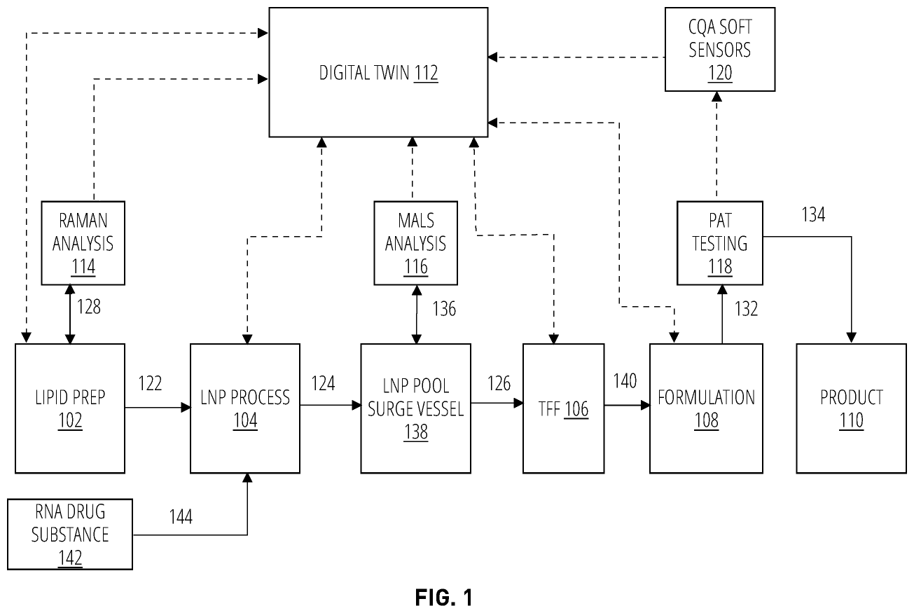

FIG. 1 is a block diagram illustrating a lipid nanoparticle (LNP) formulation portion of a continuous biomanufacturing process to provide a bulk LNP drug product 110, such as an LNP encapsulated mRNA therapeutic. As illustrated, the LNP formulation portion of the process comprises four sequential unit operations, Lipid Prep 102 (CP1), LNP 104 (CP2), TFF 106 (CP3), and Formulation 108 (CP4). Note that CP stands for critical point or critical control point (CCP). These include a lipid prep 102 step where multiple lipids are prepared in solution with a solvent such as ethanol and mixing together to form a solution (Lipid Prep), an LNP process 104 (LNP) in which an aqueous RNA fluid stream and the lipid solution fluid stream are mixing together to form lipid nanoparticles (LNPs) that encapsulate the RNA transcripts, a filtration operation (TFF), such as a tangential flow filtration operation TFF 106, and a formulation 108 operation (Formulation) in which excipients are added to produce the final drug product formulation. Although not illustrated, the formulation operation is typically followed by a sterile filtration operation and a final fill and finish operation to produce the finished drug product in vials.

In accordance with an exemplary aspect of the methods described here, product CQAs are continuously or intermittently measured at up to four points in a continuous process that includes unit operations CP1, CP2, CP3, and CP4. As illustrated in FIG. 1, these four points are located at each of the sequential unit operations of lipid prep 102 (CP1), LNP process 104 (CP2), TFF 106 (CP3) and a formulation 108 (CP4). In-line PAT instruments are connected, e.g., via a recirculation loop, at each process point. Although not illustrated, the in-line PAT instruments may be connected to each unit operation via a recirculation loop with an optional hold vessel situated in fluid communication between two consecutive unit operations in the process, e.g., between CP1 and CP2, between CP2 and CP3, and/or between CP3 and CP4.

Beginning with the first of the four sequential continuous process unit operations, at CP1 the lipid components are mixed by the lipid prep 102 unit in predetermined amounts or ratios adapted to achieve predetermined properties of the lipids in accordance with methods known in the art of lipid nanoparticle formulation technology. In accordance with this process, operating parameters of the lipid mixing process are controlled by a controller. For example, different lipid components may be fed into a vessel of the lipid prep 102 unit at a specified flow rate, which may also be referred to as an addition rate, and the vessel may comprise a means for mixing the lipids, such as an impeller, which is operated via a controller. The lipids may be fed into the mixing vessel either simultaneously or in a predetermined sequence or pattern.

In one aspect, a Raman spectroscopy instrument, which may also be referred to as a Raman PAT, is connected in-line to the lipid prep 102 unit via a recirculation loop 128, which may be referred to interchangeably as a recirculation line. In aspects, an optional hold vessel (not shown in FIG. 1) may be positioned in fluid communication between the lipid prep 102 unit and the next sequential unit operation in the continuous process, i.e., the CP2 unit, LNP process 104, such that the Raman PAT is connected in-line to the optional hold vessel via a recirculation loop. In another aspect, the Raman PAT is configured to acquire spectra via a probe situated in the CP1 vessel, in which case a recirculation loop is not necessary. Optionally, a probe of the Raman PAT may be situated in the optional hold vessel.

In operation, one or more lipid mixture CQAs are determined based on Raman spectra acquired by the Raman PAT. The one or more lipid CQAs are determined by a method including acquiring Raman spectra of a process fluid and converting the spectra via operation of a preconfigured model into one or more lipid CQA values. The one or more lipid CQA values may include total lipid concentration value and a concentration value for one or several different lipid components of the mixture such as a cholesterol concentration value, a phospholipid concentration value, such as a distearoylphosphatidylcholine (DSPC) concentration value, an ionizable cationic lipid concentration value, such as a C12-200 concentration value, and/or a polyethylene glycol (PEG) lipid derivative concentration value, such as a C14-PEG2000 concentration value. Other ionizable lipids may include DLin-MC3-DMA, SM-102, and ALC-0315. The Raman spectra may be acquired as process fluid flows through a Raman flow cell or via a Raman probe tip inserted into a vessel containing process fluid, such as a hold vessel as described above, or a unit operation such as the CP1 lipid prep mixing vessel. The Raman spectra may be acquired during or after addition of all lipid components. The one or more lipid CQA values may be determined following a predetermined time of mixing, or monitored continuously. In either case, the one or more lipid CQA values are determined and compared to a predetermined set of criteria for the lipid mixture to determine whether the criteria are satisfied. When the predetermined set of criteria for the lipid mixture are satisfied, a controller actuates a transfer mechanism to transfer the lipid mixture to the next unit operation in the process, the CP2 LNP process 104 unit, for nanoparticle formulation with drug product. The transfer of the lipid mixture may be performed via transfer line 122. For example, the controller may actuate one or more valves or pumps configured to transfer the lipid mixture from the CP1 unit to the CP2 LNP process 104 unit. In general, in accordance with the methods described here, soft sensors are configured based on parameters identified using a partial least squares (PLS) regression algorithm and calibrated with off-line analytics, such as HPLC measurements.

The Raman PAT includes at least one flow cell or probe configured for acquiring Raman spectra from a process fluid. In aspects, including aspects other than where the probe is positioned within a vessel of the unit operation itself, the fluid may be returned after spectra acquisition either to the unit operation or to a hold vessel via a fluid recirculation loop. In aspects, the Raman PAT may also include a tubing assembly for formation of a recirculation loop in fluid connection either with a unit operation of the process or with a hold vessel positioned in fluid communication between two unit operations, such as between CP1 and CP2. The tubing assembly may include flow components to provide motive force and/or control the flow of the sample, such as valves, pumps, or other equipment.

At the CP2 LNP process 104 unit operation, the drug product, for example an mRNA-based therapeutic such as an mRNA vaccine, is formulated into nanoparticles with the lipid component. RNA drug substances may be transferred to the LNP process 104 unit from an RNA drug substance 142 process or vessel via transfer line 144. The drug product may also include low molecular weight organic compounds or other forms of nucleic acids, including other forms of RNA such as small interfering RNA (siRNA). In accordance with one aspect, CQA values at the LNP process 104 unit or a subsequent LNP pool surge vessel 138 are measured in-line using dynamic light scatter (DLS) data and/or multiangle light scattering (MALS), such as that collected by a DLS or MALS detector. CQA values determined via DLS and/or MALS at the CP2 LNP process 104 unit or a subsequent LNP pool surge vessel 138 may include those related to nanoparticle physical properties, including particle size and size distribution as measured by polydispersity index (PDI), morphology, and encapsulation efficiency as measured by Ribogreen™ fluorescence assay.

The LNP process 104 operation may include a vessel, inlets, outlets, agitators, temperature control devices, or any other process equipment required to produce the lipid nanoparticles encapsulating the drug product. Exemplary techniques include high-pressure homogenization, sonication, thin film hydration methods, and nanoprecipitation methods including microfluidic techniques.

In aspects, the optional LNP pool surge vessel 138 is positioned in fluid communication between the LNP process 104 unit and the TFF 106 unit. When conditions are satisfied, a controller actuates a transfer mechanism in order to transfer the process fluid between the LNP process 104 unit and the LNP pool surge vessel 138 via transfer line 124. In certain examples, the LNP process 104 unit transfers process fluid directly to the TFF 106 unit.

A MALS and/or DLS detector is connected in-line to the LNP pool surge vessel 138 via a recirculation line 136. In an alternate example, the MALS and/or DLS detector is connected in-line to the LNP process 104 unit via a recirculation line. In either case, one or more LNP CQA values are determined via MALS and/or DLS. The one or more LNP CQA values are determined by a method including acquiring dynamic light scattering and/or multi-angle light scattering data of a process fluid and converting the data via operation of a preconfigured model into one or more LNP CQA values. The one or more LNP CQA values may include PDI and size. The data may be acquired during or after addition of all components. In alternate examples, a Raman analysis 115 may be performed in addition to the MALS and/or DLS analysis. The one or more LNP CQA values may be determined following a predetermined time of mixing or monitored continuously. In either case, the one or more LNP CQA values are determined and compared to a predetermined set of criteria for the drug encapsulated nanoparticles in order to determine whether the criteria are satisfied. When the predetermined set of criteria are satisfied, a controller actuates a transfer mechanism in order to transfer the nanoparticles to the TFF 106 unit operation for purification via transfer line 126. For example, the controller may actuate one or more valves or pumps configured to transfer the nanoparticles to the TFF 106 unit.

In accordance with an aspect, the one or more LNP CQA values are determined by a method including acquiring dynamic light scattering (DLS) and/or multi-angle light scattering (MALS) data of a process fluid and converting the data via operation of a preconfigured model into one or more LNP CQA values, where the CQA values include lipid nanoparticle size and PDI of encapsulated mRNA in lipid nanoparticles, evaluating the CQA values against a set of predetermined CQA criteria for the process using a statistical process control algorithm, and finally actuating, via a controller, the transfer of process fluid to a downstream unit operation when the criteria are met, thereby enabling the real-time release of the LNP collection pool to progress forward to the subsequent CP3 TFF 106 unit. This represents a previously unavailable approach of releasing the batch or continuous product after a unit operation is complete or has reached steady state without the use of off-line analytics.

AT CP3, the process fluid is transferred to a tangential flow filtration (TFF) unit which may include a TFF filter unit and a hold vessel into which process fluid is collected post-TFF. In one optional aspect, a Raman PAT (not pictured) is connected in-line to the CP3 TFF 106 unit via a recirculation loop. In another aspect, the Raman PAT is connected in-line to a post-TFF hold vessel such that the Raman PAT samples process fluid from this TFF pool. In aspects, the post-TFF hold vessel may be positioned in fluid communication between the CP3 TFF 106 unit and the next sequential unit operation in the continuous process, CP4 formulation 108, via transfer line 140 such that the Raman PAT is connected in-line to the hold vessel via a recirculation loop. In another aspect, a probe of the Raman PAT may be situated in the post-TFF hold vessel. In operation, one or more LNP CQA values are determined post-TFF in the TFF pool based on Raman spectra acquired by the Raman PAT. In accordance with this aspect, the critical quality attributes of mRNA-LNP constructs are monitored during and after the TFF 106 unit operation to ensure that total lipid and individual lipid component concentration values remain consistent with predetermined criteria during LNP production. In addition, the methods described herein provide indirect measurements of total mRNA concentration as determined via total mRNA input, total particle count, and encapsulation efficiency (EE) models. Total particle count is obtained with MALS, and an encapsulation efficiency model is being developed through analysis of unique Raman spectral signatures. Thus, the one or more LNP CQA values are determined post-TFF in the TFF pool based on the Raman spectra and compared to a predetermined set of criteria for the TFF pool. Where the criteria are met, a controller actuates a transfer mechanism in order to transfer the lipid mixture to the next unit operation in the process, the CP4 formulation 108 unit. As discussed above, the transfer mechanism may include actuation of one or more valves or pumps configured to transfer process fluid from the TFF pool to the CP4 formulation 108 unit. In one aspect, where the system reaches a steady state of filtration that meets the predetermined criteria for the product CQAs, transfer of process fluid from CP3 to CP4 may be continuous. The methods described here enable real-time release of the TFF collection pool to the CP4 formulation 108 unit operation. This represents a previously unavailable approach of releasing the batch or continuous flow after a unit operation is complete or at steady state without the use of off-line analytics.

With reference to FIG. 1, the last sequential unit operation in the LNP formulation process is the CP4 formulation 108 unit, in which excipient compounds are added. The formulation 108 unit may include a surge vessel or other components to receive and mix excipient additions or other products. Various spectroscopic instruments may be connected in-line at CP4 in order to determine product CQAs related to final product composition. For example, near-IR or mid-IR may be configured to determine a concentration of sucrose or ethanol; Raman spectroscopy may be utilized again at this stage, as at CP2 to determine CQAs related to concentration of mRNA lipids in the drug product; and DLS and/or MALS data may be used to determine nanoparticle size and polydispersity index (PDI) of encapsulated mRNA. In each case, CQA soft sensors 120 monitor and determine whether a set of predetermined criteria are satisfied for real-time release to the final sterile filtration, fill and finish unit operations, depicted in FIG. 1 as product 110 via transfer line 134. This represents a previously unavailable approach of releasing the batch or continuous flow after a unit operation is complete or at steady state without the use of off-line analytics.

A control module of the CQA soft sensors 120 may use the partial least squares (PLS) regression algorithm and off-line analytics to generate models using the dynamic light scatter (DLS), near infrared (Near IR), UV, and Fourier-transform infrared (FTIR, mid-IR) data. The CQA soft sensors 120 may use PLS regression models that will be calibrated using off-line analytical measurements. This process ensures the specificity of the models to the molecules of interest. The PLS models use principal component analysis to find which parameters contribute to the most variations in the Raman spectral data (X space), which will be correlated to the off-line reference values, such as high-performance liquid chromatography (HPLC) measurements (Y space). In other examples, this processing of the data may be performed at the digital twin 112 or another computing device.

In accordance with the in-line process monitoring at each of the unit operations described above, the system may also provide an alert where one or more CQAs are determined to be outside of a predetermined range.

### Digital Twin

In addition to CQA soft sensors, a system incorporating the in-line process analytic testing discussed above may also include an LNP formulation digital twin 112. Data collected by the analytical instruments and the CQA soft sensors 120 is transmitted to a computer such as a server or a set of servers executing the models of the digital twin 112 via any suitable digital or analog signal transmission technology such as via a signal cable, Wi-Fi, Bluetooth, or any other technology, indicated in the figure by dotted lines. The digital twin 112 incorporates data-driven modeling as well as mechanistic modelling to model and predict CQA values and critical process parameters (CPPs) for the various unit operations and provides monitoring and control of the entire manufacturing process. Advanced use of the digital twin 112 includes process predictive maintenance, anomaly and fault detection and sensor failure handling.

In aspects, the system may include digital twins of CP 2 LNP, CP3 TFF3, CP4 excipient addition, and final sterile 0.2 μm filtration, that utilize data-driven modeling, e.g. PLS regression modeling, as well as mechanistic differential equations to trend and predict the CQAs and CPPs for each unit operation. Based on the trends and predictions of the digital twin 112, soft sensors are utilized to alter operating parameters for optimizing the yield, quality, and safety of the final drug product.

In aspects, the mechanistic modelling of the digital twin 112 may include differential equations for kinetic processes. For example, a system of integro-differential equations with fitted parameters and initial and boundary conditions may be utilized to predict certain variables of the reaction including concentration of the components and CQAs including particle size, particle size distribution (PSD), polydispersity index (PDI) and encapsulation efficiency (EE).

Alternatively or in addition, a hybrid modelling approach may allow powerful machine learning tools to account for unknown phenomena when combined with the mechanistic models described here for improved modeling of the biological system and reaction processes. Data-driven modelling can utilize historical and newly collected data from the process runs make predictions on certain attributes such as yield and purity.

In specific implementations, the models of the digital twin 112 provide an understanding of lipid preparation processes and mRNA production rates during the described reactions, which is utilized to determine optimal starting concentrations of the reaction components, addition rates, temperatures, pressures and other parameters. The modeling may allow the system to trend and predict the operation of each unit operation as well as use the sensors and analytical equipment data to change operating parameters for optimizing the yield, quality, and safety.

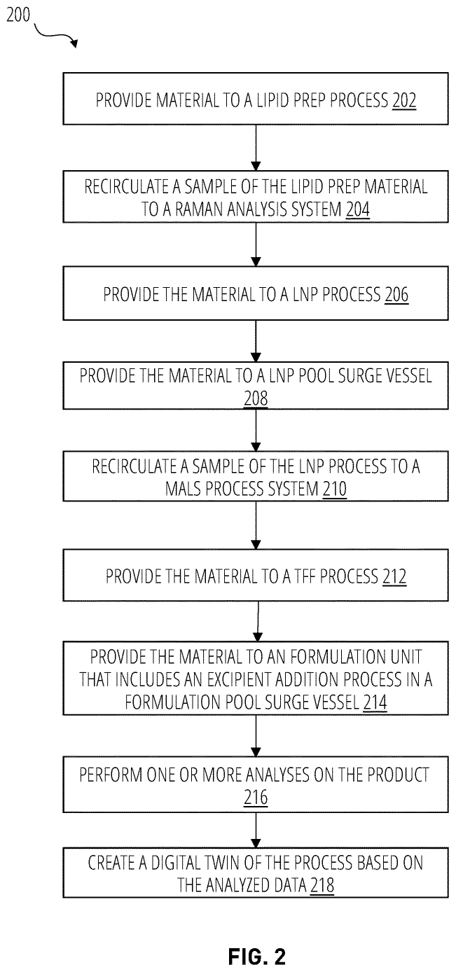

FIG. 2 is a block flow diagram illustrating an example method 200 to monitor a continuous RNA process.

In block 202, the method 200 provides a material to a lipid prep 102 process. As described herein, the lipid material may be provided to a vessel and mixed, agitated, or otherwise processed.

In block 204, the method 200 recirculates a sample of the lipid prep material to a Raman analysis 115 system. As described herein, a sample from the vessel is provided to a Raman spectroscope and/or other analyzer device for analysis. Based on the results, the material is transferrable to a subsequent processing step.

In block 206, the method 200 provides the materiel from the lipid prep 102 to the LNP process 104. In block 208, the method 200 provides the material to an optional LNP pool surge vessel 138.

In block 210, the method 200 recirculates a sample of the LNP process material to a MALS analysis 116 system from the LNP pool surge vessel 138. As described herein, a sample from the vessel is provided to a MALS device and/or other analyzer device for analysis. Based on the results, the material is transferable to a subsequent processing step.

In block 212, the method 200 provides the material to a TFF 106.

In block 214, the method 200 provides the material to a formulation 108 unit that includes an excipient addition process in a formulation pool surge vessel. Excipients are added to the material as required to meet the CQAs.

In block 216, the method 200 performs one or more analyses on the product from the formulation 108 via transfer line 132. The analyses may be performed in a PAT testing 118 system and may include one or more different analysis devices, such as a Raman spectroscope, a MALS device, Near IR, UV/VIS spectrophotometer, or any other suitable device. Based on the results, the material may be provided as a suitable product.

In block 218, the method 200 creates a digital twin of the process based on the analyzed data from one or more of the analysis devices from the method 200. The digital twin 112 may create a model that may be used to trend and predict the operation of each unit operation as well as use the sensors and analytical equipment data to change operating parameters for optimizing the yield, quality, and safety.

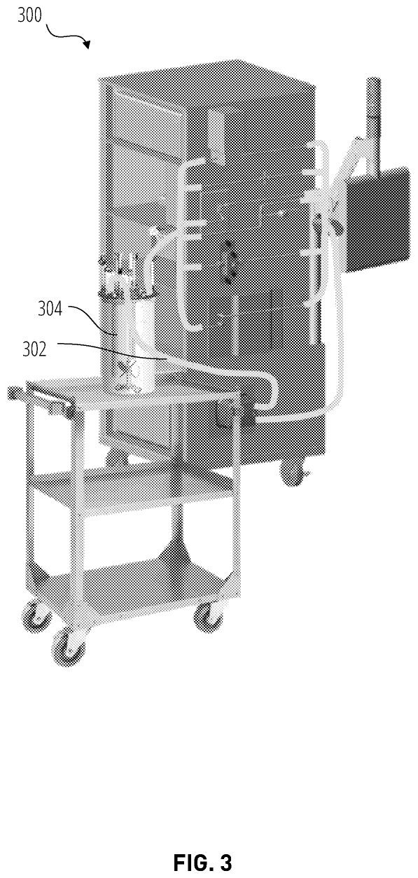

In aspects, the Raman PAT and other process analytic instruments may be housed in a compact modular skid adapted for positioning near any unit operation in a biopharmaceutical manufacturing process, as illustrated in FIG. 3.

In an aspect, provided is an apparatus for monitoring and control of an LNP formulation process including unit operations CP1, CP2, CP3, and CP4, which refer to a lipid prep 102 or lipid mixing operation (CP1), an LNP process 104 operation (CP2) in which the drug product is formed into nanoparticles with the four individual lipid components, a filtration operation 106 (CP3), such as a tangential flow filtration operation TFF 106, and a formulation 108 operation (CP4) in which excipients are added to produce the final drug product formulation. An optional CP5 unit operation includes a sterile filtration process, e.g., a 0.2 micron filtration. In accordance with this aspect, the apparatus includes Raman spectroscopy instruments connected in-line via tubing and flow cells to formulated drug product (FDP) and bulk drug product (BDP) in-process containers. The Raman instruments and associated soft sensors and controllers are adapted to monitor the concentration of the mRNA and its critical quality attributes (CQA) as well as total lipid concentration and the concentration of individual lipid components throughout the LNP formulation process.

In an exemplary embodiment, the apparatus is in the form of a compact modular skid containing the Raman spectroscopy instruments, and optionally other analytics, connected to a process vessel 304 by connection tubing 302, as illustrated FIG. 3. The apparatus may include any type of pumps, valves, hoses, conditioning equipment, flow cells, or other hardware or software to extract the sample, prepare the sample for analysis, perform the analysis, and optionally return the sample to the process.

Other process analytical instruments that may be included on the skid are a multi-angle light scattering (MALS) instrument, a dynamic light scattering (DLS) instrument, a near IR spectrometer, an IR spectrometer, and a UV/VIS spectrometer.

In aspects, provided is a method of measuring a concentration of total lipid and individual lipid components of an LNP (such as ionizable lipid, phospholipid, cholesterol, and a polyethylene glycol (PEG) lipid derivative) in a process fluid, the method comprising acquiring Raman spectra from the process fluid and converting the spectra into one or more lipid concentration values by implementing a PLS model developed from a library of predetermined spectra of known offline lipid concentration values.

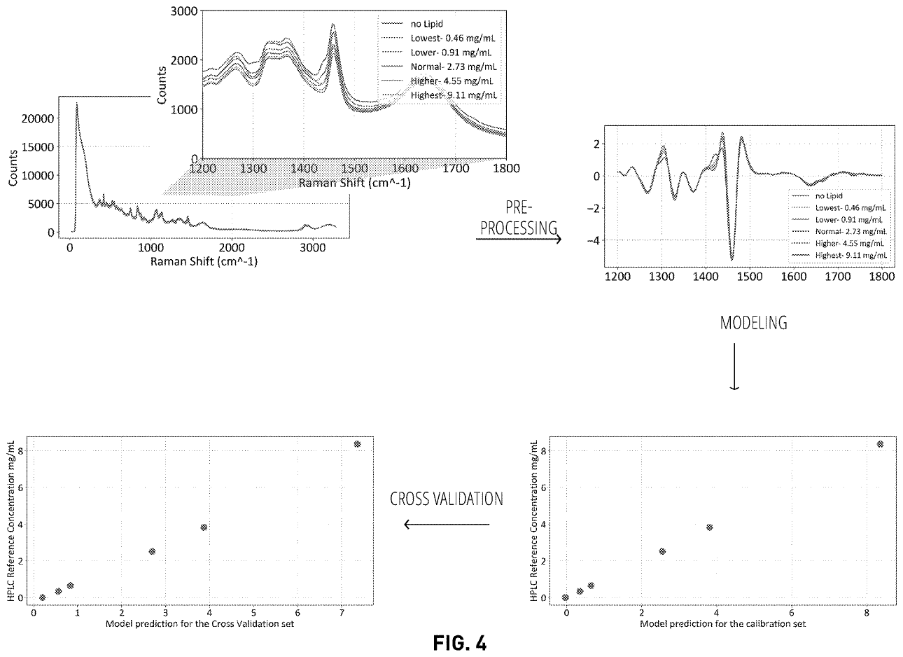

FIG. 4 illustrates modeling of total lipid concentration from Raman spectra acquired from samples containing known concentrations of lipid. An initial PLS model was trained using off-line T-mixer samples and calibrated using off-line HPLC measurements. This model was then used to predict the total lipid concentration during additional runs by feeding Raman data to the model in real-time. The Raman instrument collected one measurement per two minutes. The data was fed into the PLS model in real-time and model predictions were generated in real-time. Additional off-line samples were collected during the run to validate the model predictions.

The portion of the Raman spectra highlighted for analysis includes the region from 1200-1800 cm⁻¹. The various lines in the inset indicate varying concentrations of total lipid, including 0 (no lipid), 0.46 mg/ml, 0.91 mg/ml, 2.73 mg/ml, 4.55 mg/ml and 9.11 mg/ml. The raw spectra are pre-processed, for example using a Savitzky-Golay smoothing and 2nd derivation filter, and normalized before being trained for PLS modeling. The two dot plots show the HPLC reference concentration versus model prediction for the calibration set and the cross-validation set, both of which demonstrate excellent linearity.

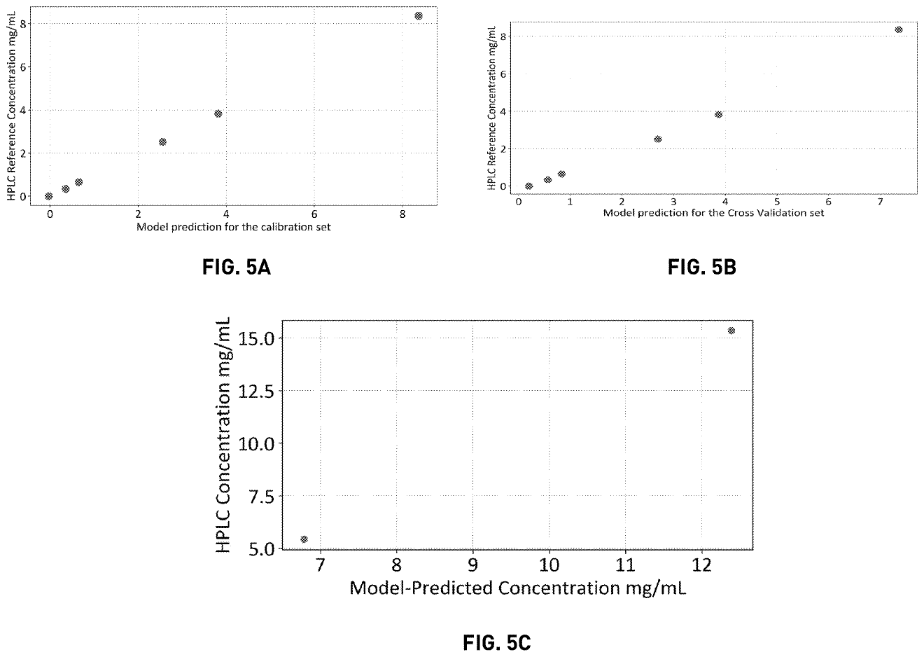

FIG. 5A shows reference lipid concentration (mg/ml) as measured by HPLC versus the model prediction for a calibration set. In this example, the model performed with an R²=0.99, Q²=0.98, RMSE=0.02, and RMSEcv=0.44. FIG. 5B shows reference lipid concentration as measured by HPLC versus the model prediction for a cross-validation set. FIG. 5C shows lipid concentration as measured by HPLC versus the model prediction for a drug product in CP1 during two independent runs, run 9 and run 11. As shown in the table below, the model performed well in predicting the total lipid concentration at CP1.

TABLE 1
Testing Total Lipid Concentration Model on CP1 Runs

| | CP1-9 | CP1-11 |
|---|---|---|
| HPLC | 5.44 | 15.35 |
| Predicted (avg) | 6.78 | 13.52 |
| RMSE | 1.37 | 1.85 |

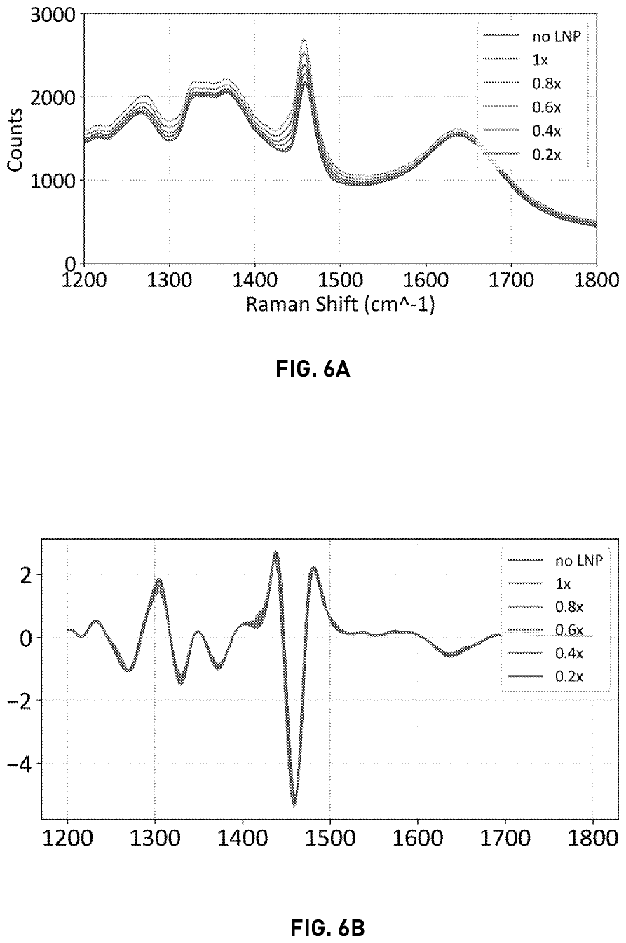

The model was also tested and validated using LNP dilutions, demonstrating its utility in quantifying the total particle count. FIG. 6A illustrates the raw Raman spectra of LNPs produced with a T-mixer, following a freeze-thaw cycle, in a focused area from 1200-1800 cm⁻¹ for a series of dilutions ranging from 1 to 0.2× of a stock solution. Without knowing the exact particle count (which is obtained through MALS), a reference value relative to the stock solution was used. Specifically shown are spectra for 1×, 0.8×, 0.6×, 0.4× and 0.2× dilutions, with relative particle counts of 1, 0.8, 0.6, 0.4, and 0.2. FIG. 6B shows the same spectra following pre-processing. FIG. 6C shows the reference concentration versus model prediction for the calibration set and FIG. 6D shows the same for the cross-validation set. The model performance parameters were R²=0.99, Q²=0.98, RMSE=0.00088, and RMSEcv=0.05.

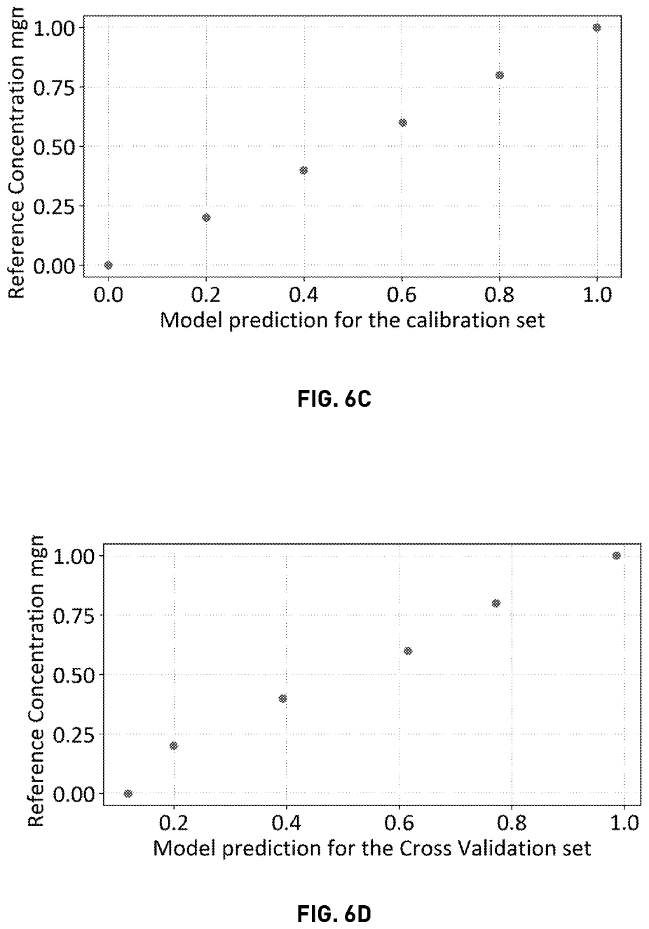

Raw materials spectra of the individual lipid components (ionizable lipid, phospholipid, cholesterol, and a polyethylene glycol (PEG) lipid derivative) were measured using Raman spectroscopy in order to develop the model used to forecast the amount of each lipid component present in a nanoparticle. By identifying the unique spectral signature of these lipid components, the process is able to develop a model to measure the concentration of these components within an LNP. In this study, individual lipids were initially dissolved in ethanol to simulate the medium the lipid stock solution resides in before mixing, but ethanol has a strong background signal overpowering the Raman spectral signal of the individual lipids. Next, the study tested the raw spectra of dry powdered lipid components, except the ionizable lipid. The ionizable lipid is provided as a viscous oil and was measured as-is. FIG. 7A shows the raw spectra of dry powder lipid components including the ionizable lipid, cholesterol, distearoylphosphatidylcholine (DSPC), and a polyethylene glycol (PEG) lipid derivative. FIG. 7B shows the pre-processed spectra where pre-processing included smoothing, 2nd derivation filter and standard normal variate normalization, but no baseline correction or subtraction.

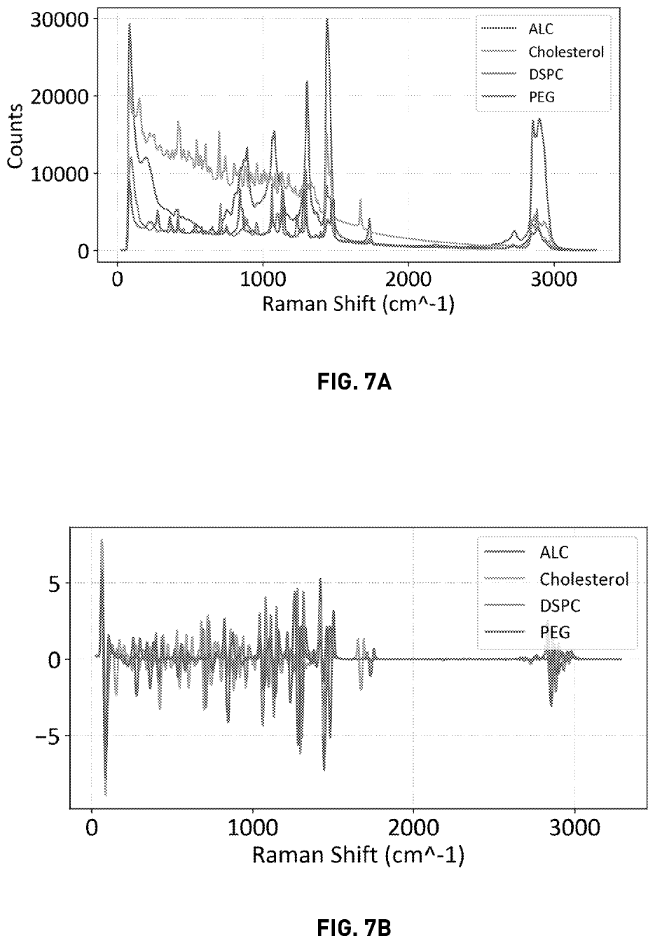

Due to the overlapping nature of the Raman spectra of each of the lipid components, the process seeks to identify unique spectral regions associated with each lipid component. Thus, a loadings analysis was performed to direct lipid component modeling using various lipid stock solution concentrations above and below the standard lipid concentration used for LNP production. These lipid stock solutions were examined and analyzed to understand the equipment detection limitations. Loadings values represent the correlation between predictor variable, i.e. Raman shift values, and latent variables, i.e. components generated from the PLS model. This indicates that the peaks of a loadings plot highlight the spectral regions (i.e. Raman shift regions) that have a significant impact on the model. Thus, a PLS model specific to each lipid component can be generated specifically at these regions identified by the loadings plot. FIG. 8A shows a selected Raman spectral region of from 1200-1800 cm⁻¹. Component lipid peaks appear at 1700 (DSPC, PEG), 1140 (Cholesterol) and 1673 (Cholesterol).

FIG. 8B shows a selected Raman spectral region of 2700-3000 cm⁻¹ containing the lipid vibrational signatures, e.g., C—H bond at 2884 and CH₂ stretching at 2760, 2830, 2850, and 2860. These spectral regions were identified as described in Bera et al., Scientific Reports 14, Article no: 4348 (2024), using Raman imaging of mRNA LNP vaccines.

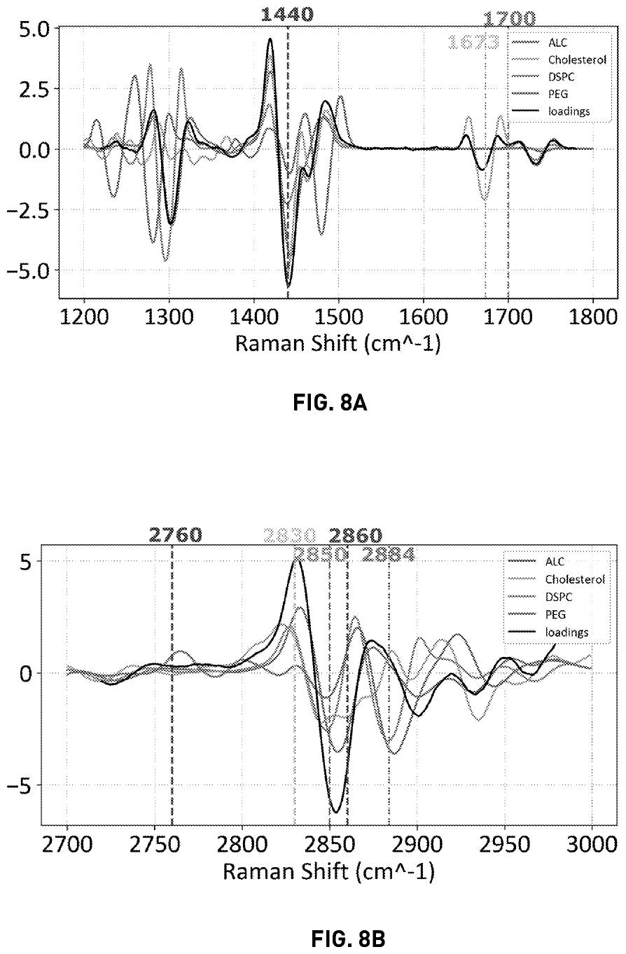

The next two figures illustrate how loadings analysis shows model specificity towards individual lipid components. FIG. 9 shows a selected Raman spectral region from 1400-1500 cm⁻¹. FIG. 9A shows a peak at 1419 cm⁻¹ for the ionizable lipid (ALC) and cholesterol; FIG. 9B shows a peak at 1323 cm⁻¹ for the ionizable lipid; and FIG. 9C shows a cholesterol peak at 1671 cm⁻¹. FIG. 9D shows this information integrated into a model spectra.

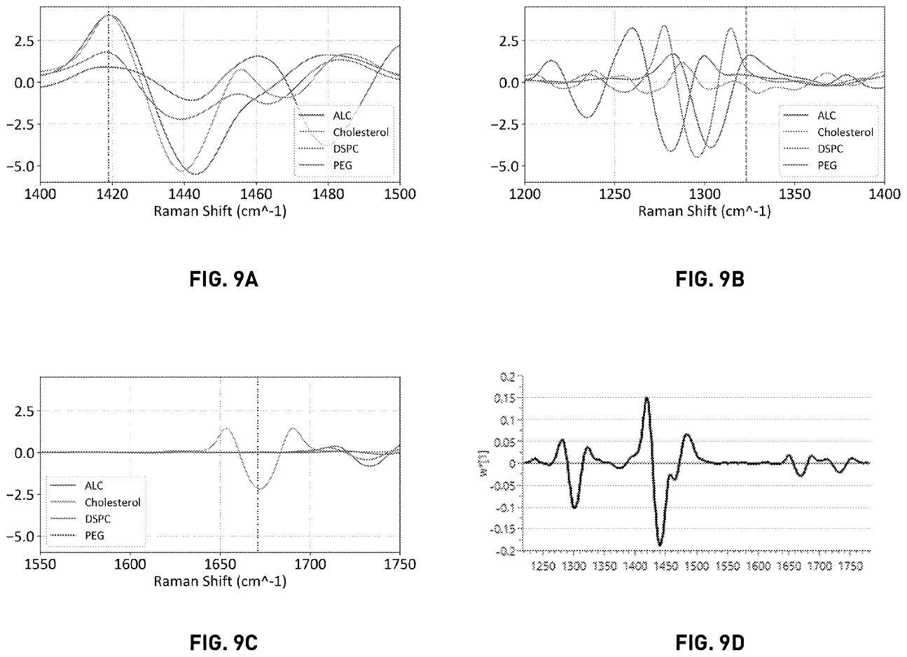

Similarly, FIGS. 10A-10C show a selected Raman spectral region from 2700-3000 cm⁻¹. FIG. 10A shows a peak at 2831 for ALC; FIG. 10B shows a peak at 2933 for cholesterol; and FIG. 10C shows a peak at 2901 for ALC and DSPC. FIG. 10D shows this information integrated into a model spectra.

Data has been collected to train models for prediction of critical quality attributes (CQAs) during the mRNA downstream production, specifically the lipid nanoparticle formulation process. These models will be used for real-time in-line continuous monitoring of the bioprocess.

A model for the lipid solution preparation (LSP) unit operation, liquid prep 102 where the lipid components are mixed in ethanol prior to the LNP formulation 104, has been developed for the digital twin 112 for predicting the following CQAs: total lipid concentration, individual lipid component concentrations (i.e. ionizable lipid, phospholipid, cholesterol, PEG).

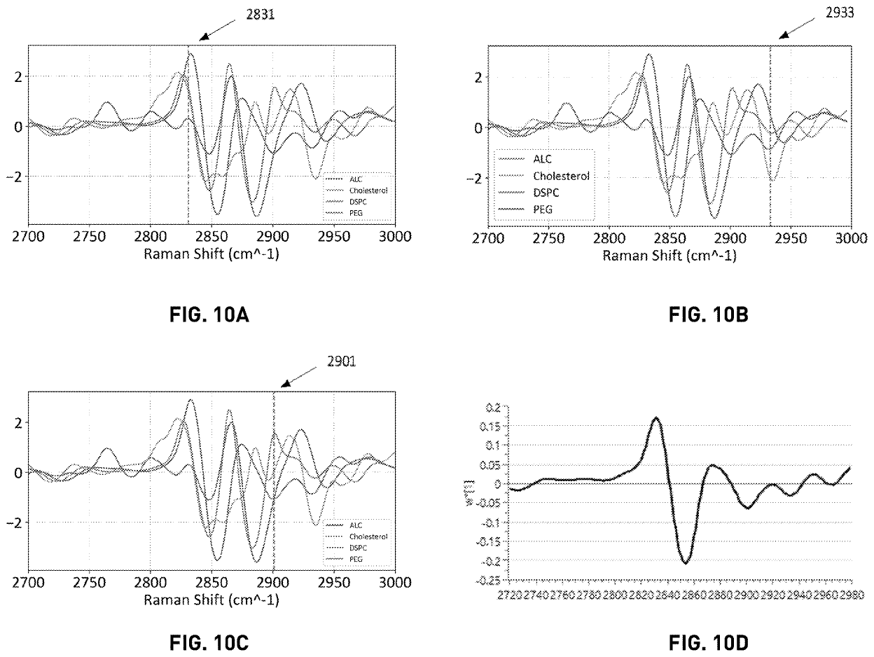

PLS regression models were developed across a broad parameter space by adjusting different spectra pre-processing and modelling parameters. These parameters include the spectral window where the model was developed, the samples used to generate the model (either all the samples, i.e. “Native”, or residual samples where the background ethanol signature was subtracted from each spectra, i.e. “Residual”), the pre-processing functions applied (Savitsky-Golay smoothing and filtration, standard normal variate, or both), and the number of components used in the PLS regression model. A panel of model permutations were generated and evaluated in order to identify optimal model conditions that maximize model performances for each CQA. The heatmap in FIG. 11 summarizes Q² values (the cross-validated coefficient of determination), a stringent quantification of model performance, across 24 model variants for the total lipid concentration prediction in the LSP pool unit operations, spanning combinations of two spectral windows (1000-1600 cm⁻¹ and 2800-3000 cm⁻¹). The model performance was assessed with the cross-validated coefficient of determination (Q²).

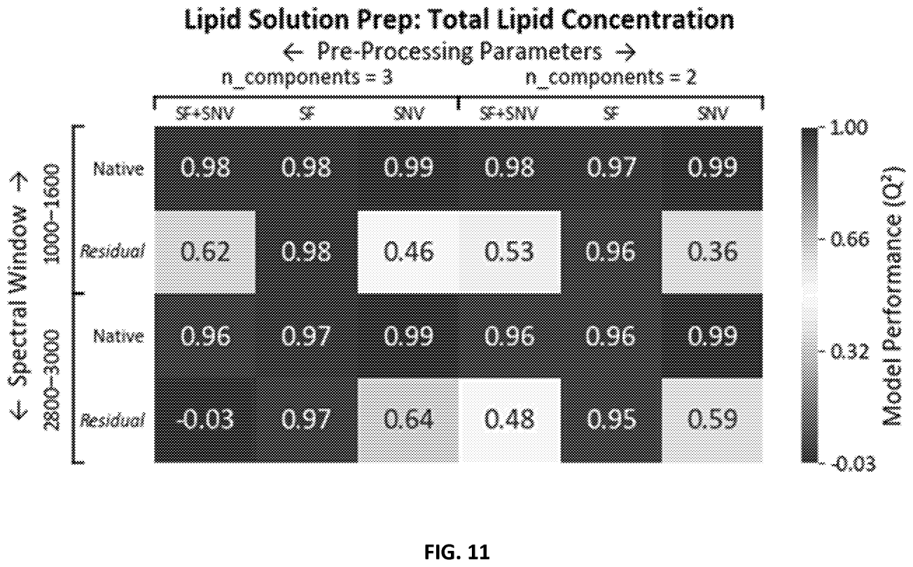

Following parameter optimization, the best-performing model configurations (correlating to a linear cross-validation predicted vs. reference concentration plot and low prediction errors) were identified for each CQA and evaluated by cross-validation. FIG. 12 summarizes the PLS models that demonstrated the best predictive performance for the following CQAs:
Total Lipid Concentration: Native spectra, SNV pre-processing, 2800-3000 cm⁻¹ spectral range, n_components=2; Q²=0.99, RMSEcv=0.75 mg/mL
Ionizable Lipid Concentration (ALC): Native spectra, SF+SNV pre-processing (SF window length=41), 1000-1800 cm⁻¹ spectral range, n_components=3; Q²=0.98, RMSEcv=1.03 mg/mL
Cholesterol: Native spectra, SF+SNV pre-processing (SF window length=41), 1600-1750 cm⁻¹ spectral range, n_components=3; Q²=0.99, RMSEcv=0.21 mg/mL
DSPC: Residual spectra, SF pre-processing (SF window length=15), 650-750 cm⁻¹ spectral range, n_components=3; Q²=0.95, RMSEcv=0.27 mg/mL

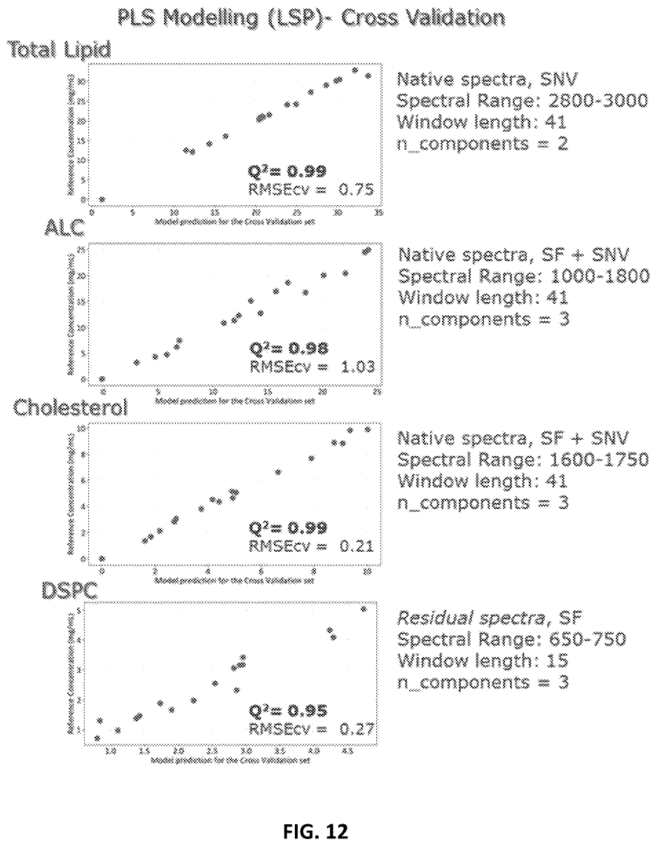

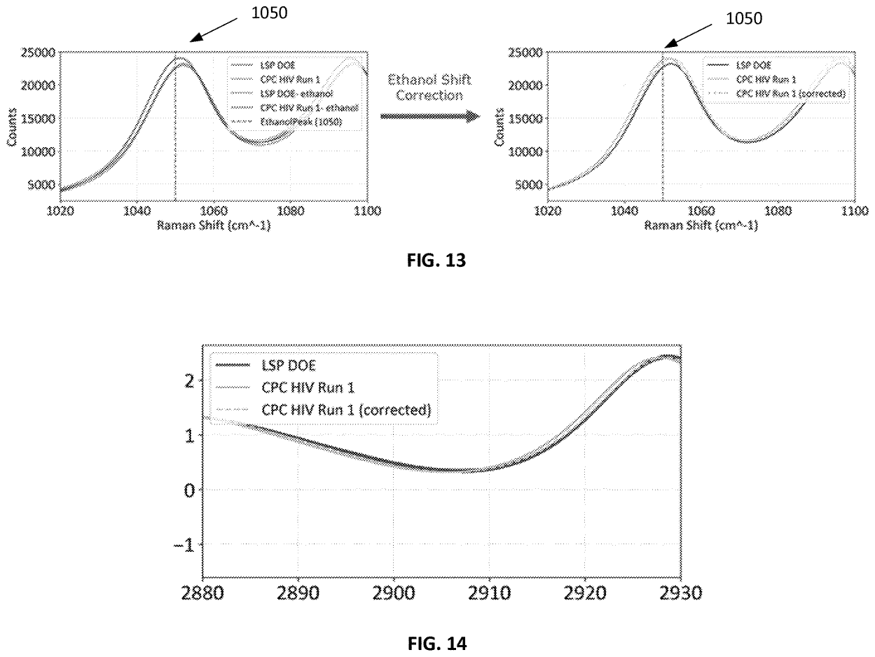

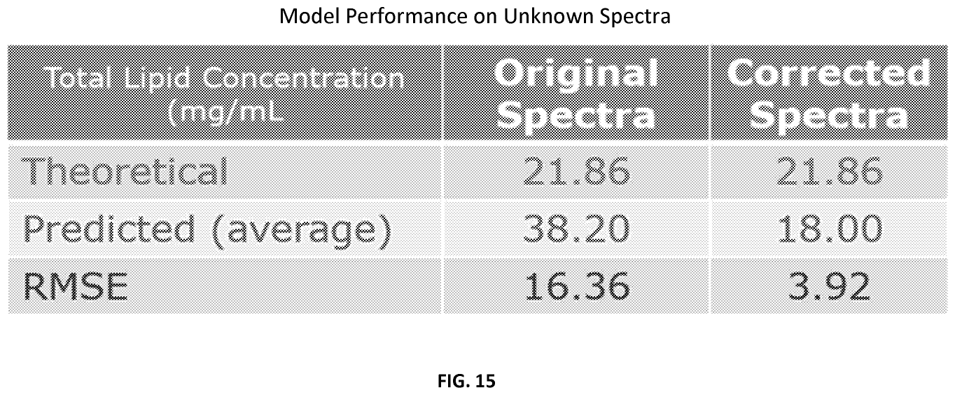

To demonstrate the suitability of these models for real-time inline monitoring of lipid CQAs during the LSP Mixing unit operation, we tested these models on data generated during this step from prior runs. We validated the model using new spectra from a process run (CPC HIV Run 1) as the prediction set. Challenges arise with model validation due to spectral shifts that occur between the model and prediction spectra. An ethanol shift was identified at a prominent peak (1050 cm\hat{ }-1), resulting in a Raman shift of 1; this shift was applied to the raw spectra of the prediction dataset, shown in FIG. 13. The “LSP DOE” (LSP design of experiments) was used to generate the model and “CPC HIV Run 1” was the prediction spectra on which the model was tested. This resulted in a slight shift in the pre-processed spectra, aligning better with the pre-processed model spectra (shown in FIG. 14). Table 2 in FIG. 15 shows the model performance using the prediction spectra from the process run, comparing the “Original Spectra” with the ethanol-shifted “Corrected Spectra” of the theoretical, predicted, and the root mean square error (RSME) for the total lipid concentration (mg/mL).

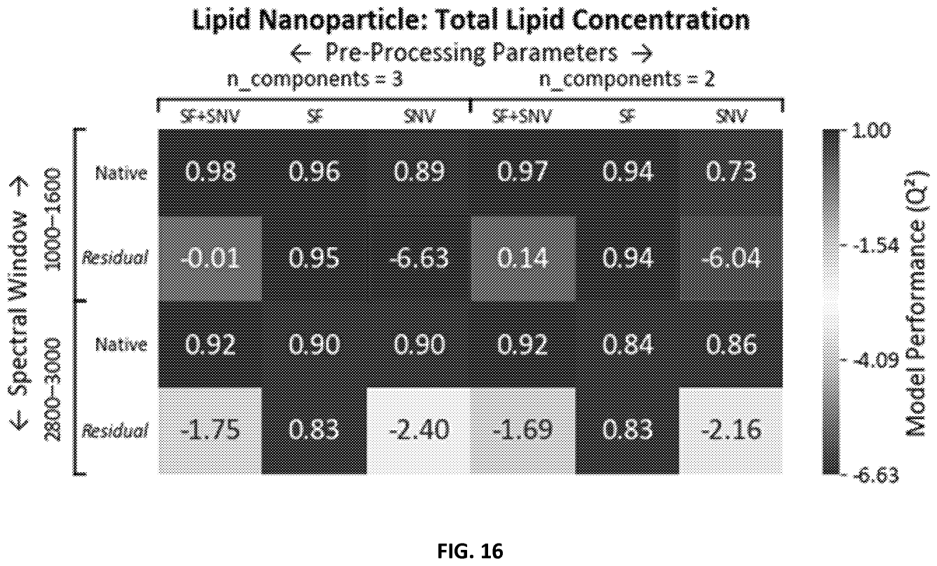

A model for the lipid nanoparticle (LNP) unit operation where the drug product is mixed with the lipid solution prep mix to form a drug product encapsulated in lipid nanoparticles, followed by tangential flow filtration (TFF) and excipient (i.e. tris/sucrose) addition to produce the final drug product formulation. A subset of this work was shown previously, and we have since generated a panel of PLS regression models across the same broad parameter space used for the LSP model development. FIG. 16 summarizes the PLS models that demonstrated the best predictive performance for the following CQA:
Total Lipid Concentration: Native spectra, SF+SNV pre-processing, 1200-1800 cm⁻¹ spectral range, n_components=3; Q²=0.98, RMSEcv=0.44 mg/mL

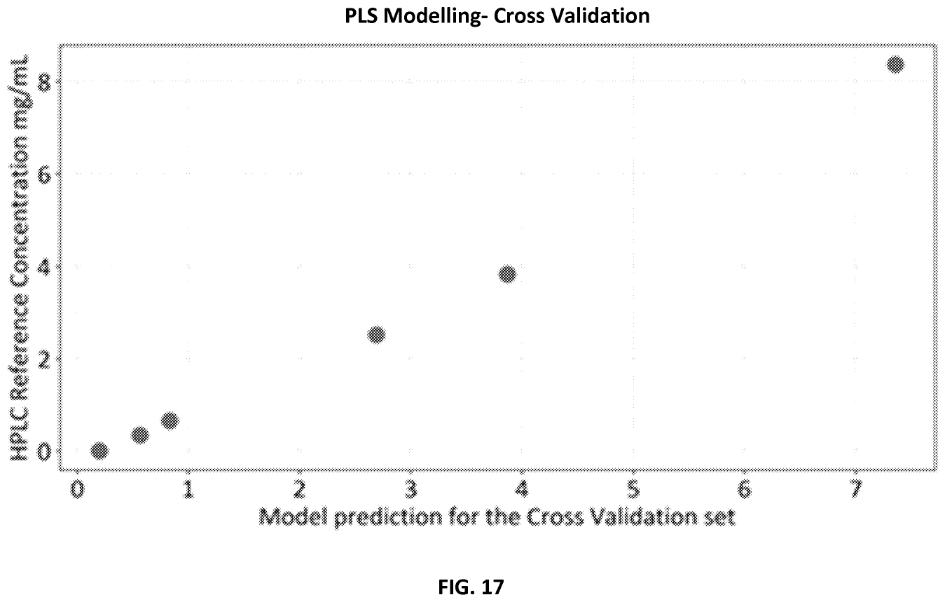

FIG. 17 shows the reference concentration versus model prediction for the cross-validation set for the best performing model.

While the invention herein disclosed has been described by means of specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

Various features of a process system may be used independently of, or in combination, with each other. It will be appreciated that a system as disclosed herein may be embodied in different forms and should not be construed as limited to the illustrated embodiments of the figures.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A compact modular apparatus for continuous process lipid nanoparticle (LNP) formulation comprising:
a Raman spectrometer and optionally an optical detector selected from one or both of a dynamic light scattering (DLS) detector and a multiangle light scatter (MALS) detector;
a tubing assembly;
an optional container external to the Raman spectrometer;
a pump; and
a controller comprising at least one soft sensor configured to receive multivariate Raman spectra data from the spectrometer and transform the data into a product critical quality attribute (CQA) selected from one or more of mRNA concentration, total lipid concentration, and component lipid concentration.

2. The apparatus of claim 1, wherein the component lipid concentration includes a concentration value for one or more of a phospholipid including distearoylphosphatidylcholine (DSPC), cholesterol, an ionizable cationic lipid, comprising a C12-200, DLin-MC3-DMA, SM-102, or ALC-0315, and/or a polyethylene glycol (PEG) lipid derivative, including a C14-PEG2000.

3. The apparatus of claim 1, wherein the soft sensor includes a predictive model for estimating the CQA from the received Raman spectra data.

4. The apparatus of claim 3, wherein the model is calibrated using off-line analytical measurements.

5. The apparatus of claim 1, wherein the apparatus comprises a DLS detector and/or a MALS detector and associated soft sensors adapted to receive and transform DLS and MALS data into a CQA.

6. The apparatus of claim 5, wherein the CQA is selected from lipid nanoparticle size, size distribution, and polydispersity index (PDI, d) of encapsulated mRNA.

7. The apparatus of claim 1, wherein the apparatus comprises a near-IR or mid-IR spectrometer and associated soft sensor adapted to receive and transform IR spectral data into a CQA.

8. The apparatus of claim 7, wherein the CQA is a sucrose or ethanol concentration value.

9. A system to produce a final drug product formulation via continuous process lipid nanoparticle (LNP) formulation, comprising:
an apparatus comprising a Raman spectrometer and optionally an optical detector selected from one or both of a dynamic light scattering (DLS) detector and a multiangle light scatter (MALS) detector, a tubing assembly, an optional container external to the Raman spectrometer, a pump, and a controller comprising at least one soft sensor configured to receive multivariate Raman spectra data from the spectrometer and transform the data into a product critical quality attribute (CQA) selected from one or more of mRNA concentration, total lipid concentration, and component lipid concentration; and
four sequential unit operations, CP1, CP2, CP3, and CP4, wherein CP1 is a lipid mixing operation, CP2 is a lipid nanoparticle formulation operation in which a drug product is mixed with the lipids of CP1 to form drug product encapsulated in lipid nanoparticles, CP3 is a tangential flow filtration operation and CP4 is an excipient formulation operation in which excipients are added to produce the final drug product formulation.

10. The system of claim 9, wherein the system further comprises one or more digital twins configured to trend and predict product attributes and process parameters at one or more of the unit operations.

11. A method for in-line continuous monitoring and control of a lipid nanoparticle formulation process, the method comprising
acquiring a set of Raman spectral data from a process fluid at a lipid mixing unit operation, CP1,
transforming the Raman spectral data, via operation of a pre-established computational model of a soft sensor, into a lipid concentration value,
comparing, via operation of a controller, the lipid concentration value to a predetermined range of lipid concentration values preloaded into the controller, and
performing an operation, via the controller, selected from hold and release of the process fluid from CP1 to the next unit operation in the lipid nanoparticle formulation process, CP2.

12. The method of claim 11, wherein the process fluid comprises a mixture of lipids and the lipid concentration value is selected from a total lipid concentration value and a concentration value of one or more component lipids in the mixture.

13. The method of any one of claim 11, wherein the one or more component lipids comprises one or more of a phospholipid, such as distearoylphosphatidylcholine (DSPC), an ionizable cationic lipid, such as a C12-200, DLin-MC3-DMA, SM-102, and ALC-0315, and/or a polyethylene glycol (PEG) lipid derivative, such as a C14-PEG2000.

14. The method of any one of claim 11, wherein the set of Raman spectral data comprises data of raw Raman spectra in a region of 1200-1800 cm⁻¹ and the lipid concentration value is a total lipid concentration value.

15. The method of any one of claim 11, wherein the set of Raman spectral data comprises data of raw Raman spectra in a region of 1200-1800 cm⁻¹ and in a region of 2700-3000 cm⁻¹ and the lipid concentration value is a concentration value of one or more component lipids in the mixture.

16. The method of claim 15, wherein the computational model utilizes an entirety of the regions identified (i.e. 1200-1800 cm⁻¹ and 2700-3000 cm⁻¹) to develop a model for total lipid concentration, wherein individual lipid component concentrations utilize characteristic bands incorporating the regions identified from a loadings analysis in which:
characteristic bands of DSPC are at 2901 cm⁻¹;
characteristic bands of cholesterol are at 2933 cm⁻¹, 1419 cm⁻¹, and 1671 cm⁻¹,
characteristic bands of ionizable lipid are at 1323 cm⁻¹, 2831 cm⁻¹, and 2901 cm⁻¹; and
characteristic bands of PEG are at 1700 cm⁻¹.

17. The method of any one of claim 11, wherein the computational model utilizes characteristic bands at 1419 cm⁻¹, 1323 cm⁻¹, and 1671 cm⁻¹ to determine a concentration value of the ionizable lipid, ALC-0315, and/or cholesterol.

18. The method of any one of claim 11, wherein the computational model utilizes a partial least squares (PLS) regression algorithm.

19. The method of claim 11, further comprising creating, by a processor of a digital twin, a model of the lipid mixing unit operation, CP1.

20. The method of claim 19, wherein the method further comprises trending and predicting, by a processor the model of the digital twin, the lipid mixing unit operation, CP1.

21. The method of claim 11, further comprising:
acquiring a set of dynamic light scatter (DLS) spectral data and/or multiangle light scattering (MALS) spectral data from a process fluid comprising mRNA, lipids, empty lipid nanoparticles, and lipid nanoparticles encapsulating mRNA; and
transforming the DLS and/or MALS spectral data, via operation of a second computational model, into one or more lipid nanoparticle attributes selected from nanoparticle size, nanoparticle size distribution, and polydispersity index (PDI) to determine one or more attributes of the lipid nanoparticles in the process fluid.

22. The method of claim 11, further comprising:
acquiring a set of dynamic light scatter (DLS) spectral data and/or multiangle light scattering (MALS) spectral data from a process fluid at the lipid nanoparticle formulation unit operation, CP2, wherein the process fluid at CP2 comprises mRNA, lipids, empty lipid nanoparticles, and lipid nanoparticles encapsulating mRNA,
transforming the DLS and/or MALS spectral data, via operation of a second computational model, into one or more lipid nanoparticle attributes selected from nanoparticle size, nanoparticle size distribution, and polydispersity index (PDI),
comparing, via operation of the controller, the one or more lipid nanoparticle attribute values to a predetermined range of lipid nanoparticle attribute values preloaded into the controller, and
performing an operation, via the controller, selected from hold and release of the process fluid from CP2 to the next unit operation in the lipid nanoparticle formulation process, CP3.
